(19) United States
(12) Patent Application Publication
(10) Pub. No.: US 2011/0229589 A1
(43) Pub. Date: Sep. 22, 2011

(54) INSECT AND PLANT DISEASE CONTROL COMPOSITIONS AND METHODS OF USE THEREOF

(76) Inventor: Hanan ELRAZ, Kiryat Bialik (IL)

(21) Appl. No.: 13/117,156

(22) Filed: May 27, 2011

Related U.S. Application Data
(63) Continuation-in-part of application No. PCT/IB2009/055378, filed on Nov. 27, 2009.

(51) Int. Cl. A01N 65/28; A01N 65/22; A01P 1/00; A01P 7/00; A01P 3/00; B82Y 5/00

(52) U.S. Cl. 424/742; 424/745; 977/773

(57) ABSTRACT

A combination of plant essential oils in an aqueous solution containing vinegar provided a pesticidal effect against a range of pests, including fungi, bacteria, oomycetes, insects such as mosquitoes and bedbugs, and nematodes, and uses thereof.

This application is a Continuation-in-Part of, and claims priority from, PCT Application No. PCT/IB2009/055378, filed on Nov. 27, 2009, which claims priority from U.S. Provisional Application No. 61/118,441, filed on Nov. 27, 2008; and from U.S. Provisional Application No. 61/350,486, filed on Jun. 2, 2010; all of which are hereby incorporated by reference as if fully set forth herein.

### FIELD OF THE INVENTION

The present invention relates to the field of insect and plant disease control compositions, and more particularly to a natural insect and plant disease control composition comprising a combination of at least two essential oils and vinegar.

### BACKGROUND OF THE INVENTION

Commercial cultivation of plants is a major part of the economy, encompassing not only crops grown for human food and animal feed, but also those, like cotton, grown for fiber, and others, such as flowers, grown for beauty. The importance of plants to people and to the economy can hardly be overstated. Plants are, however, also subject to constant attack by insects, fungi, oomycetes, bacteria, viruses, nematodes, and other pathogens. When pathogens find susceptible plants, these attacks can result in the loss of yield and quality, and may result in the loss of entire crops. These losses result in substantial economic harm to the growers and, in some areas of the world, contribute to famine.

The majority of phytopathogenic fungi belong to the Ascomycetes and the Basidomycetes. Significant plant pathogens include the Ascomycetes Fusarium, Thielayiopsis; Verticillium; Magnaporthe grisea, and the Basidiomycetes Rhizoctonia, Phakospora pachyrhizi; and Puccinia.

The oomycetes are not true fungi, but are fungal-like organisms. Despite not been closely related to the fungi, the oomycetes have developed very similar infection strategies and so many plant pathologists group them with fungal pathogens. Significant oomycete plant pathogens include Phythium and Phytophthora.

Nematodes are small, multicellular wormlike creatures, which may infect plant roots. Potato cyst nematodes are widely distributed in Europe and North and South American and cause millions of dollars worth of damage in Europe every year.

Plants may also be affected by insects, which may feed on leaves; feed on and into fruit, seeds and nuts; feed on and tunnel into roots; tunnel or bore into stems, stalks, branches and trunks; suck the sap from leaves, stems, roots, fruits and flowers; and transmit plant disease agents.

Mosquito control is also highly important; mosquitoes and other insects, such as bed bugs, in the worst case scenario transmit many human diseases, but even in the best case scenario are extremely annoying and impact human quality of life. Previously, sprays featuring DDT (dichlorodiphenyltrichloroethane) were used to kill such insects, while treatments of human skin with DEET (N,N-diethyl-3-methylbenzamide or N,N-diethyl-m-toluamide) helped to repel them. However both substances are of highly questionable safety. Unfortunately safer alternatives are typically not effective.

### SUMMARY OF THE INVENTION

The present invention, in at least some embodiments, provides an insect and plant disease control formulation comprising tea tree oil in an amount of from about 5% to about 35% volume per volume over the volume of the formulation; an emulsifier in an amount of up to about 10% volume per volume; a vinegar in an amount of up to about 40% volume per volume and a further essential oil selected from the group consisting of citronella, rosemary oil, lemon grass oil and neem oil, each present in an amount of from about 5% to about 35% volume per volume, or a combination of two or more of said further essential oils; wherein a solvent carrier comprises the balance of the formulation.

According to at least some embodiments, there is provided an insect and plant disease control formulation comprising citronella in an amount of from about 10% to about 22%, volume per volume over the volume of the formulation; Lemon grass oil in an amount up to 10%; Vinegar in an amount of up to 5% volume per volume; Rosemary oil in an amount of from about 10% to about 22%, volume per volume; and an emulsifier in an amount of up to 3% volume per volume, wherein a solvent carrier comprises the balance of the formulation.

Without wishing to be limited in any way, the formulation has been found to be particularly useful for treatment and control of agriculturally important diseases, and control of insects, including with regard to insects that present a hazard to human health or discomfort (such as mosquitoes and bugs).

As used herein, the term “control” also relates to prevention of such diseases and insects, and their extermination. Prevention of insects relates to prevention of insect infestation, whether with regard to repelling or extermination in any stage of their life cycle or in multiple stages. The insect and plant disease control formulation is also referred to herein as a “pesticide”; surprisingly, as described in greater detail below, the natural pesticide formulation described herein is both effective against insects and plant diseases, yet is not harmful to the surrounding environment or to humans, plants or animals, unlike synthetic pesticides.

According to some embodiments, the natural pesticide of the present invention is used for control of agriculturally important diseases, including but not limited to those diseases caused by a fungus (such as Rhizoctonia, Pythium, Verticillium dahlia, or fusarium oxysporum), an oomycete (such as Phythium or Phytophthora), a bacterium (such as streptomyces) or a nematode.

According to some embodiments, the natural pesticide of the present invention is useful for control of insects such as tetranychidae (including spider mite, red spider mite, oriental red mite, fruit tree red spider mite, and European red spider mite), aphididae (including melon aphids and cotton aphids), eriosomatidae (such as wooly aphids), aleyrodidae (such as sweet potato whitefly), diaspididae (such as California red scale), thysanoptera; pterygota (such as thrips) and agromyzidae (such as serpentine leafminer), as well as for control of grain and seed crop pests including but not limited to members of the lissorphotrus family such as Lissorhoptrus oryzophilus (rice water weevil), members of the tribolium family such as Tribolium castaneum (flour beetle), and members of the sitophilus family such as Sitophilus oryzae (rice weevil).

According to other embodiments, the formulation is useful for repelling and/or controlling insects which provide a hazard to human health or are otherwise problematic for human sanitation. Non-limiting examples of such insects include Culex pipiens (English name: House mosquito); Culex univittatus; Aedes mariae; anopheles, blackfly, tsetse fly, Phlebotomus and Cimex lectularius (bedbugs).

The novel combination of ingredients in the insect and agricultural disease control formulation of the present invention creates a substantially enhanced effect compared to that obtained by using each of the ingredients separately.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

Where ranges are given, endpoints are included within the range. Furthermore, it is to be understood that unless otherwise indicated or otherwise evident from the context and understanding of one of ordinary skill in the art, values that are expressed as a range can assume any specific value or subrange within the stated range in different embodiments of the invention, to the tenth of the unit of the lower limit of the range, unless the context clearly dictates otherwise. Where a percentage is recited in reference to a value that intrinsically has units that are whole numbers, any resulting fraction may be rounded to the nearest whole number.

In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-1C are bar graphs showing the effect of different concentrations of the formulation of the present invention on nematodes in tomato plants. FIG. 1A shows the fresh shoot weight; FIG. 1B shows the galling index; FIG. 1C shows the number of eggs in the roots; and

FIGS. 2A-2E show nematodes after treatment with different concentrations of the formulation of the present invention (FIGS. 2A-2C); with control (FIG. 2D); or with a commercial anti-nematode product (FIG. 2E).

### DESCRIPTION OF THE PREFERRED EMBODIMENTS

The present invention provides a novel, all natural insect and plant disease control formulation which is environmentally friendly.

The present invention is based on the surprising finding that a combination of plant essential oils in an aqueous solution containing vinegar provided a pesticidal effect against a range of pests, including fungi, bacteria, oomycetes, insects such as agriculturally damaging insects (such as mites and aphids), mosquitoes and bugs and nematodes. However, a further surprising finding is that this pesticidal effect does not negatively impact plants, humans, ecological system or the general environment, nor does it negatively impact important and desirable insects such as bees or the natural enemies of the target pests.

As used herein, a ‘pesticide’ is a composition which destroys, prevents, repels or mitigates any pest, including insects, fungi, oomycetes, bacteria, nematodes, viruses, viroids, virus-like organisms, phthoplasmas, and protozoa.

As used herein, an ‘essential oil’ is any concentrated, hydrophobic liquid containing volatile aroma compounds from plants. Essential oils in the context of the invention may include, without limitation, an essential oil selected from neem oil, lemongrass oil, citronella oil, eucalyptus oil, tea tree oil, lavender oil, spearmint oil, geranium oil, rosemary oil, lemon balm oil, peppermint oil, pine needle oil, lavandin oil, cinnamon oil, clove oil, thyme oil, wintergreen oil, cedar oil, lemon oil, grapefruit oil, mandarin oil, tangerine oil, orange oil, citrus oil, lime oil, coriander oil, pomegranate oil and cajeput oil.

As used herein, ‘vinegar’ refers to a sour, aqueous liquid, generally containing about 4%-8% acetic acid, obtained by the acetic fermentation of dilute, aqueous alcohol solutions, e.g. by bacterial fermentation of wine, apple cider or fruit juice. Preferably, the vinegar comprises apple vinegar.

As used herein, the term ‘aqueous formulation’ refers to a water-based composition, wherein the major component of the composition, by volume, is water.

As used herein, the term ‘active ingredient’ refers to a compound which possesses one or more pesticide functions when applied to a crop and/or which possesses one or more insect control functions.

As used herein, the term ‘emulsifier’ refers to a substance which stabilizes an emulsion, frequently a surfactant. Non-limiting examples include Tween 20 (polysorbate 20) and paraffin oil.

In some embodiments, the formulation further comprises additional ingredients, such as, for example, sugars, vitamins, and plant growth factors which can be absorbed by the roots and leaves of growing plants. These factors are preferably present in the essential oils as described with regard to Example 16 below.

According to at least some embodiments of the present invention, the compositions or formulations as described herein may optionally be used, or adapted for use, as an insect control measure for environmental locations. Such environmental locations include, without limitation, any type of outdoor location, including, without limitations, agricultural fields, landscaped areas, other types of fields and grounds, ponds, lakes, rivers, puddles and other bodies of water, outdoor holding pens or coops for livestock, poultry and the like; and also any type of indoor location, including without limitation hothouses, greenhouses, barns, chicken coops or barns, livestock pens and the like.

By “insect control” it is meant reducing the numbers of insects, whether as adults or immature forms (including without limitation eggs, pupae and larvae). Such reduction may optionally occur through killing the insects at any stage or more than one of the above stages (wherein each form relates to a different stage in the life cycle of the insect), by reducing the successful reproduction rate and/or by repelling the insects at any one or more stages from the area in which the composition is distributed. Any type of distribution may optionally be used, including without limitation, spraying, spreading, coating, dripping, dissolving and the like, for powder, liquid and foam compositions. Solid compositions may also optionally be distributed in any suitable manner, and may also optionally be dissolved in liquid at the area of distribution. Any suitable carrier may optionally be included to form the composition, for example and without limitation, any type of aqueous carrier, oily carrier, or emulsion (such as oil in water or water in oil emulsions). Producing the various forms of the compositions could easily be performed by one of ordinary skill in the art.

Active Formulation

The insect and plant disease control formulation described herein preferably comprises an active formulation, comprising at least two essential oils and vinegar (preferably but not limited to apple vinegar), with an emulsifier.

The active formulation, in at least some embodiments, comprises tea tree oil in an amount of from about 5% to about 35% volume per volume over the volume of the formulation; an emulsifier in an amount of up to about 10% volume per volume; a vinegar in an amount of up to about 40% volume per volume and a further essential oil selected from the group consisting of citronella, rosemary oil, lemon grass oil and neem oil, each present in an amount of from about 5% to about 35% volume per volume, or a combination of two or more of said further essential oils.

Preferably, the tea tree oil is present in an amount of from about 10% to about 22%, volume per volume. More preferably, said further essential oil comprises citronella present in an amount of from about 10% to about 22%, volume per volume; said emulsifier is present in an amount up to about 3%, said vinegar is present in amount of up to about 10%.

According to other embodiments, said further essential oil comprises lemon grass oil and rosemary oil. Optionally and preferably, lemon grass oil is present in an amount up to about 10% volume per volume and said rosemary oil is present in an amount of from about 10% to about 22%, volume per volume, while said emulsifier is present in an amount up to about 3% and said vinegar is present in amount of up to about 5%.

According to still other embodiments, and said further essential oil comprises citronella and neem oil. Optionally and preferably, citronella is present in an amount of from about 10% to about 22% volume per volume, said emulsifier is present in an amount up to about 3%, said vinegar is present in amount of up to about 10% and said neem oil is present in an amount up to about 5% volume per volume.

According to still other embodiments, said further essential oil comprises rosemary oil, lemongrass oil and neem oil. Optionally and preferably, rosemary oil is present in an amount of from about 10% to about 22%, volume per volume, said lemon grass oil is present in an amount of up to about 10%, said emulsifier is present in an amount up to about 3%, said vinegar is present in amount of up to about 5% and said neem oil is present in an amount up to about 5% volume per volume.

According to still other embodiments, the active formulation is formulated such that the further essential oil comprises citronella or rosemary oil, and said further essential oil is present in an amount equal to said tea tree oil.

According to yet other embodiments, the active formulation comprises Tea tree oil in an amount of from about 10% to about 22%, volume per volume over the volume of the formulation; Citronella in an amount of from about 10% to about 22%, volume per volume; Vinegar in an amount of up to 10% volume per volume; and an emulsifier in an amount of up to 3% volume per volume.

According to yet other embodiments, the active formulation comprises Tea tree oil in an amount of from about 10% to about 22%, volume per volume over the volume of the formulation; Lemon grass oil in an amount up to 10%; Vinegar in an amount of up to 5% volume per volume; Rosemary oil in an amount of from about 10% to about 22%, volume per volume; and an emulsifier in an amount of up to 3% volume per volume.

According to yet other embodiments, the active formulation comprises Tea tree oil in an amount of from about 10% to about 22%, volume per volume over the volume of the formulation; Citronella in an amount of from about 10% to about 22%, volume per volume; Vinegar in an amount of up to 10% volume per volume; neem oil in an amount of up to 5% volume per volume; and an emulsifier in an amount of up to 3% volume per volume.

According to yet other embodiments, the active formulation comprises Tea tree oil in an amount of from about 10% to about 22%, volume per volume over the volume of the formulation; Lemon grass oil in an amount up to 10% volume per volume; Rosemary oil in an amount of from about 10% to about 22%, volume per volume; neem oil in an amount of up to 5% volume per volume; Vinegar in an amount of up to 5% volume per volume; and an emulsifier in an amount of up to 3% volume per volume.

According to yet other embodiments, the active formulation comprises citronella in an amount of from about 10% to about 22%, volume per volume over the volume of the formulation; Lemon grass oil in an amount up to 10%; Vinegar in an amount of up to 5% volume per volume; Rosemary oil in an amount of from about 10% to about 22%, volume per volume; and an emulsifier in an amount of up to 3% volume per volume.

Specific non-limiting examples of formulations are given below.

### Formula 1

Tea tree oil: 10%-22%

### Citronella: 10%-22%

### Vinegar: up to 10%

### Tween 20—up to 3%

Rest: water

### Formula 2

Lemon grass oil: up to 10%
Tea tree oil: 10%-22%

### Rosemary oil: 10%-22%

### Paraffin: up to 3%

### Vinegar: up to 5%

Rest: water

### Formula 3

Tea tree oil: 10%-22%

### Citronella: 10%-22%

### Vinegar: up to 10%

### Tween 20—up to 3%

### Neem oil: up to 5%

Rest: water

### Formula 4

Lemon grass oil: up to 10%

### Rosemary oil: 10%-22%

Tea tree oil: 10%-22%

### Neem oil: up to 5%

### Paraffin: up to 3%

### Vinegar: up to 5%

Rest: water

### Formula 5

Lemon grass oil: up to 10%
citronella: 10%-22%

### Rosemary oil: 10%-22%

### Paraffin: up to 3%

### Vinegar: up to 5%

Rest: water

Liquid Carrier

As described herein, according to at least some embodiments of the present invention, the formulation optionally and preferably features a liquid carrier. The liquid carrier may optionally comprise one or more solvents in which the active formulation dissolved or solubilized; alternatively or additionally, the liquid carrier may optionally comprise one or more liquids in which the active formulation is suspended.

Optionally a solvent carrier comprises the balance of the formulation.

Optionally and preferably, the solvent carrier comprises water, and wherein said water is present in an amount of up to 70% volume per volume over the formulation.

Any suitable liquid carrier may optionally be included to form the composition, for example and without limitation, any type of aqueous carrier, oily carrier or emulsion (such as oil in water or water in oil emulsion). Producing the various forms of the composition could easily be performed by one of ordinary skill in the art.

As described herein, the term “carrier”, whether liquid or solid, denotes an organic or inorganic ingredient, natural or synthetic, with which the active ingredient is combined to facilitate its application, for example, to the plant, to seeds, to the soil or to aquatic surroundings. This carrier is therefore generally inert and it must be acceptable (for example, agronomically acceptable, particularly for a treated plant).

The carrier may optionally be any suitable liquid, for example: water; alcohols, particularly butanol or glycol, as well as their ethers or esters, particularly methylglycol acetate; ketones, particularly acetone, cyclohexanone, methylethyl ketone, methylisobutylketone, or isophorone; petroleum fractions such as paraffinic or aromatic hydrocarbons, particularly xylenes or alkyl naphthalenes; mineral oil; aliphatic chlorinated hydrocarbons, particularly trichloroethane or methylene chloride; aromatic chlorinated hydrocarbons, particularly chlorobenzenes; water-soluble or strongly polar solvents such as dimethylformamide, dimethyl sulphoxide, or N-methylpyrrolidone; liquefied gases; or the like or a mixture thereof.

The formulation of the present invention may optionally be distributed using any known method, including without limitation, spraying, spreading, coating, dripping, dissolving, and the like, for liquid and foam compositions.

For treating fields and other agricultural locations and environment against insects and air borne diseases, optionally and preferably a diluted form of the active formulation is sprayed with a coverage of from 80 liters to 240 liters per acre. For treating fields against soil borne diseases (and also optionally insects in the soil), optionally and preferably a diluted form of the active formulation is sprayed with a coverage of from 400 ml to 2400 ml per acre. By “air borne” disease it is meant any disease or insect infestation that is partially or completely spread and/or travels through the air for at least a portion of its life cycle. By “soil borne” disease it is meant any disease or insect infestation that is present and/or spreads through soil for at least a portion of its life cycle.

Solid Carrier

According to at least some embodiments of the present invention, any of the active formulations described herein may optionally be prepared as a dry formulation by adsorbing the formulation onto a solid carrier.

Any other suitable method for preparing such a dry formulation may also optionally be used as is known in the art, for example through wet granulation of the active formulation with an inert carrier, followed by drying.

Optionally the solid carrier comprises an inert material and in a final form preferably is selected from the group consisting of pellets, particles, microparticles and nano-particles, but may optionally also comprise a raw material such as raw compost or fertilizer. By “inert” it is meant that any activity of the solid carrier is not related to a function or activity of the active formulation.

Optionally the dry formulation in a final form preferably is in a form selected from the group consisting of pellets, particles, microparticles and nano-particles.

The carrier may optionally comprise, for example, clays, natural or synthetic silicates, silica, resins, waxes, solid fertilizers (for example ammonium salts), ground natural minerals, such as kaolins, clays, talc, chalk, quartz, attapulgite, montmorillonite, bentonite or diatomaceous earth, or ground synthetic minerals, such as silica, alumina, or silicates especially aluminium or magnesium silicates. As solid carriers for granules the following are suitable: crushed or fractionated natural rocks such as calcite, marble, pumice, sepiolite and dolomite; synthetic granules of inorganic or organic meals; granules of organic material such as sawdust, coconut shells, corn cobs, corn husks or tobacco stalks; kieselguhr, tricalcium phosphate, powdered cork, or absorbent carbon black; water soluble polymers, resins, waxes; or solid fertilizers. Other organic materials include but are not limited to bone meal, animal waste (including but not limited to urine and fecal matter) and homogenized animal tissue from any suitable animal, including but not limited to poultry and livestock; and compost (optionally pasteurized).

Such solid compositions may, if desired, contain one or more compatible wetting, dispersing, emulsifying or coloring agents which, when solid, may also serve as a diluent. Other materials which may be added include but are not limited to nitrogen, calcium, potash and the like.

The formulation of the present invention may be distributed using any known method, including without limitation, spraying, spreading, coating, dripping, dissolving, and the like, for powder and particulate compositions; pellets are typically dropped but may also optionally be sprayed.

Solid compositions may also optionally be distributed in any suitable manner, and may also optionally be dissolved in liquid at the area of distribution.

Dilutions and Diluents

The above described formulations may optionally comprise any type of liquid or dry diluent. Any of the above described carriers may optionally be used as a diluent. If a liquid diluent is used, preferably the liquid diluent dilutes the active formulation to form a total formulation such that the active formulation comprises from 0.2% to 6% and preferably from 0.5% to 4% of the total formulation.

Methods of Preparing Insect and Plant Disease Control Formulations

In another aspect, the present invention provides a method of preparing a pesticide formulation, comprising:
a. Mixing a combination of essential oils with an emulsifier;
b. Adding to the mixture, water at a temperature of about 40° C. and vinegar, wherein the balance of the formulation comprises water;
c. Mixing for about 2 hours at ambient temperatures using a mechanical stirrer; and
d. Allowing the mixture to rest prior to use, the resting period may be for about 24-72 hours, preferably for about 48 hours.

Methods of Use

The formulation of the present invention is useful for treating and preventing a wide range of agriculturally important diseases, and for preventing damage by insects, in both organic and conventional agriculture.

As used herein, the term “treating” includes abrogating, substantially inhibiting, slowing or reversing the progression of an agricultural disease and/or of an insect infestation.

According to some embodiments, the formulation of the present invention is useful for improving the yield and/or quality of a wide range of agricultural products, including fruits and vegetables such as apples, cucumbers, tomatoes, eggplants, potatoes, citrus fruit, pears, plums, apricots, avocados, bananas, pineapples, mangoes, kiwis, peaches, melons, watermelons, nectarines, strawberries, zucchini, turnips, pumpkins, lettuce, cabbage and peppers; flowers, such as roses, poppies, tulips, gypsophila, gerbera; herbs and spices, such as myrtle, cloves, basil, mint, parsley, dill; and nuts.

According to some embodiments, the formulation of the present invention, at low concentrations (0.1%-3%) is useful for post-harvest protection of agricultural produce. For example, fruit and vegetables, after harvesting, may be immersed in a bath containing the formulation and/or may optionally be sprayed or fogged with the formulation. Such treatment, according to some embodiments, reduces the deterioration rate of the produce and improves the final quality.

The formulation of the present invention is also useful for improving growth of domestic plants, such as garden and house plants.

According to some embodiments, the formulation of the present invention is useful for killing of fungi (including, for example, Rhizoctonia, Pythium, Verticillium dahlia, fusarium oxysporum); bacteria (such as streptomyces); and nematodes; as well as various insects, all of which are known to cause damage to agricultural products.

According to some embodiments, the formulation of the present invention may be used as an insect control measure for environmental locations. Such environmental locations include without limitation any type of outdoor location, including agricultural fields, landscaped areas, other types of fields and grounds, ponds, lakes, rivers, and other bodies of water, outdoor holding pens or coops for livestock, poultry and the like; and also any type of indoor location, including without limitation hothouses, greenhouses, barns, chicken coops or barns, livestock pens and the like.

By ‘insect control’ it is meant reducing the number of insects, whether as adults or immature forms (including without limitation eggs, pupae and larvae). Such reduction may optionally occur through killing the insects at any stage or more than one of the above stages (wherein each form relates to a different stage in the life cycle of the insect), by reducing the successful reproduction rate and/or by repelling the insects at any one or more stages from the area in which the composition is distributed.

Insects which are controlled by the formulation of the present invention include, but are not limited to members of the tetranychidae family, including mites such as spider mite, red spider mite, oriental red mite, fruit tree red spider mite, European red spider mite; members of the aphididae family, including aphids such as melon aphids, cotton aphids; members of the eriosomatidae family, such as wooly aphids; members of the aleyrodidae family, such as sweet potato whitefly; members of the diaspididae family, such as California red scale; members of the thysanoptera family; members of the pterygota family, such as thrips; and members of the agromyzidae family, such as serpentine leafminer.

The formulations of the present invention have been shown not to cause damage to beneficial insects used as a biological means of pest control, such as predatory mites used for the control of spider mites, sweet potato whitefly and western flower thrips; parasitic wasps for the control of leafminer flies and sweet potato whitefly; parasitic wasps and gall midge predators used for the control of aphids; predatory bugs for the control of western flower thrips; and parasitic wasps and predatory beetles for the control of mealybugs.

According to at least some embodiments of the present invention, there is provided use of a natural pesticide formulation for control of agriculturally important diseases caused by at least one of a fungus, an oomycete, a bacterium, a nematode or an insect infestation, wherein said pesticide comprises vinegar, an emulsifier and a further essential oil selected from the group consisting of citronella, rosemary oil, tea tree oil and lemon grass oil, in an aqueous carrier. Optionally and preferably, the essential oils are present in a total amount of from 10% to 30% volume per volume over the volume of the formulation. Of course, optionally any of the above described formulations may be used for these methods of treatment.

According to other embodiments, the above methods of treatment and the above described formulations may also optionally relate to repelling and/or controlling insects which provide a hazard to human health or are otherwise problematic for human sanitation. Non-limiting examples of such insects include Culex pipiens (English name: House mosquito); Culex univittatus; Aedes mariae; anopheles, blackfly, tsetse fly, Phlebotomus and Cimex lectularius (English name: bedbugs).

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

### Materials

The essential oils were each purchased from Perry & Lowe, Hamburg, Germany, and used without dilution. All-natural 5% apple vinegar (95% water) was purchased in Israel from Carmel Mizrachi.

### Formulations

Formulation 1 according to at least some embodiments of the present invention was prepared as previously described and was then diluted to the final concentration that was used.

The formulation was prepared by first forming a concentrate comprising a stock solution which is the above described active formulation. The concentrate was then mixed with distilled water to obtain the below described v/v ratios in the total diluted formulation.

### Example 1

### Effect on Rhizocatonia

The effect of the natural pesticide according to at least some embodiments of the present invention, Formulation 1, was studied on Rhizoctonia growing on potato shoots.

The formulation, at concentrations of 1%, 2% or 3% of the stock solution was shaken for 20 minutes before use. Potato shoots were immersed in the formulation for 30 minutes, with continuous shaking. Shoots were then removed and dried for 24 hours at room temperature. The process was then repeated. Following the second drying period, rigidity obtained from the potato shoots were placed on a nutrient layer in a petri dish. The number of live fungi was measured 2, 3, and 7 days after immersion in the fungicidal composition.

Results are shown in Table 1 below:

TABLE 1

| Percentage | % live fungi | % live fungi | % live fungi | |
|---|---|---|---|---|
| stock | after 2 days | after 2 days | after 2 days | % decrease |
| control | 100.0 | 100.0 | 100.00 | — |
| Formulation 1 | 8.9 | 11.1 | 11.1 | 88.89 |
| 1% | | | | |
| Formulation 1 | 8.9 | 20.0 | 20.0 | 80.00 |
| 2% | | | | |
| Formulation 1 | 6.7 | 11.1 | 11.1 | 88.89 |
| 3% | | | | |

### Example 2

### Effect on Various Fungi

The effect of the composition of the present invention, Formulation 1, was tested on the fungi Rhizoctonia, Pythium, Verticillium dahlia, fusarium oxysporum, and on Streptomyces bacteria. Results are shown in Tables 2-6 below.

TABLE 2
Rhizoctonia

| Concentration | Percentage growth |
|---|---|
| Formulation 1 | retardation relative to control |
| 1% | 100 |
| 2% | 100 |
| 3% | 100 |

Standard

| | |
|---|---|
| 0.1 ppm | 88.24 |
| 0.5 ppm | 100 |

TABLE 3
Pythium

| | Percentage growth |
|---|---|
| Concentration Formulation 1 | retardation relative to control |
| 1% | 100 |
| 2% | 100 |
| 3% | 100 |

Standard (Dionon)

| | |
|---|---|
| 1 ppm | 24.88 |
| 5 ppm | 57.78 |

TABLE 4
Verticuillium dahliae

| | Percentage growth |
|---|---|
| Concentration Formulation 1 | retardation relative to control |
| 1% | 100 |
| 2% | 100 |
| 3% | 100 |

Standard (Bevistin)

| | |
|---|---|
| 0.5 ppm | 84.55 |
| 1 ppm | 100 |

TABLE 5
Fusarium oxysporum

| | Percentage growth |
|---|---|
| Concentration Formulation 1 | retardation relative to control |
| 1% | 83.9 |
| 2% | 100 |
| 3% | 100 |

Standard (Benlate)

| | |
|---|---|
| 10 ppm | 84.55 |
| 100 ppm | 100 |

TABLE 6
Streptomyces

| Concentration Formulation 1 | CFU (colony forming units) |
|---|---|
| control | 1E+09 |
| 1% | 0 |
| 2% | 0 |
| 3% | 0 |

Standard (Bactoril)

| | |
|---|---|
| 1 ppm | 5.0E+03 |
| 10 ppm | 0 |

### Example 3

### Effect on Nematodes

The effect of the composition of the present invention, Formulation 1, at concentrations of 10, 50 and 100 ml per liter soil was tested on the nematode Meloidognye javanica. The known insecticide Nemacur EC400 (Fenamiphos, N-[ethoxy-(3-methyl-4-methylsulfanylphenoxy)phosphoryl]propan-2-amine), at a concentration of 0.5 mg per liter soil, was used as a standard.

Tomatoes (strain 144) were used, which were grown in a 700 cm³ pot containing 900 gr soil. Soil used was red loam soil. The soil infection about 2000 M. javanica per pot. Tomato plants were planted one week after treatment with the composition of the present invention, or with Nemacur EC400.

Each treatment was carried out on 7 individual pots containing tomato plant, and was accomplished through dripping, but could also have been performed through spraying, administration of pellets and so forth.

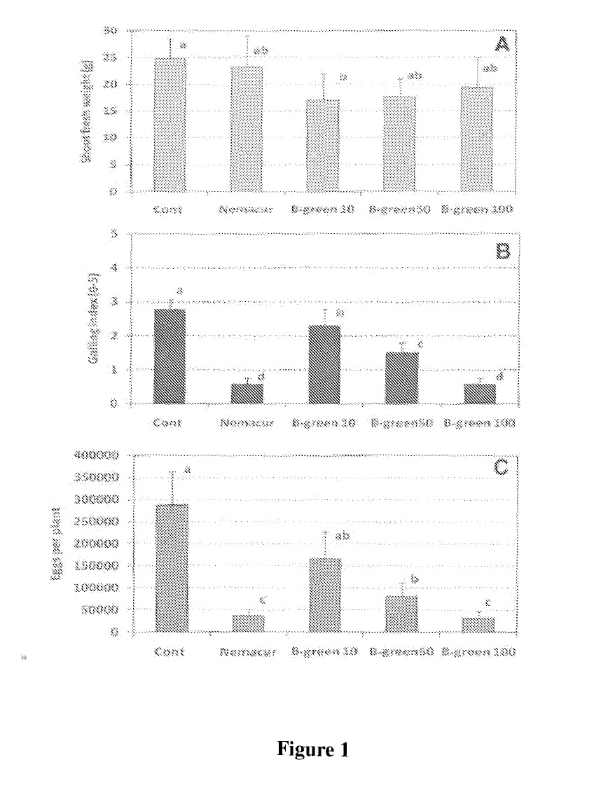

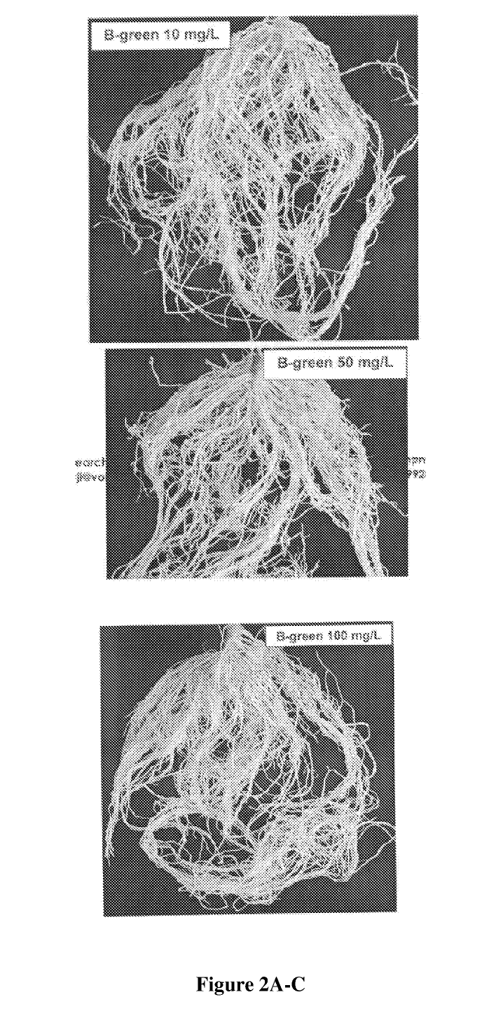

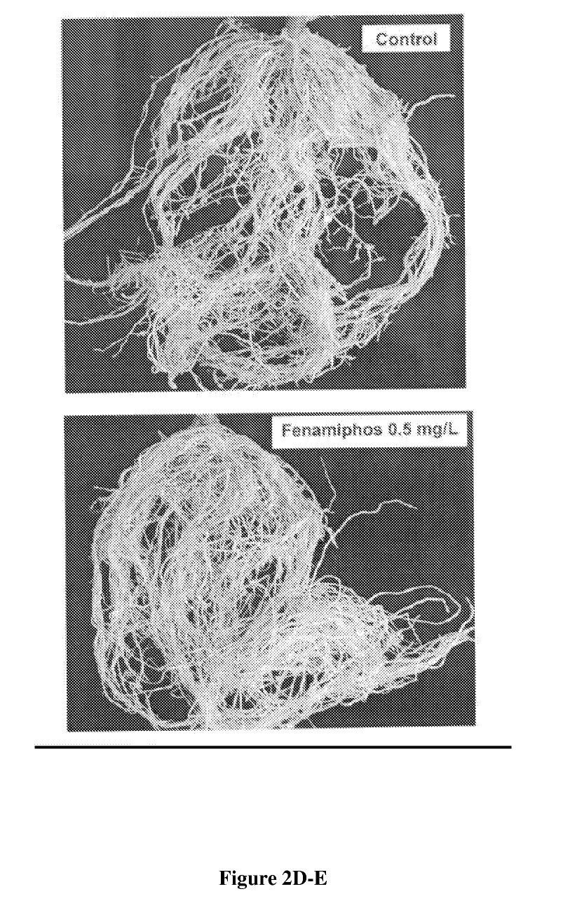

The fresh weight of the shoots, galling index in the roots (from 0 to 5), and number of eggs in the roots 6 weeks after planting was noted. Results are presented in Tables 7-9 and in FIGS. 1 and 2.

TABLE 7
Fresh weight of shoots (g)

| | Formu- | Formu- | Formu- | | |
|---|---|---|---|---|---|
| | lation 1 | lation 1 | lation 1 | | |
| | 10 ml/l | 50 ml/l | 100 ml/l | Nemacur | Control |
| Mean | 16.9 | 17.6 | 19.4 | 23.3 | 24.8 |
| SD | 5.1 | 3.4 | 5.5 | 5.6 | 3.8 |

TABLE 8
Galling index (0-5)

| | Formu- | Formu- | Formu- | | |
|---|---|---|---|---|---|
| | lation 1 | lation 1 | lation 1 | | |
| | 10 ml/l | 50 ml/l | 100 ml/l | Nemacur | Control |
| Mean | 2.3 | 1.5 | 0.6 | 0.6 | 2.8 |
| SD | 0.5 | 0.3 | 0.2 | 0.2 | 0.3 |

TABLE 9
Eggs per plant

| | Formu- | Formu- | Formu- | | |
|---|---|---|---|---|---|
| | lation 1 | lation 1 | lation 1 | | |
| | 10 ml/l | 50 ml/l | 100 ml/l | Nemacur | Control |
| Mean | 165,750 | 80,550 | 31,800 | 36,750 | 288,000 |
| SD | 61,972 | 31,107 | 16,708 | 14,427 | 75,215 |

As seen in Table 7 and FIG. 1A, the results showed a decrease in the fresh weight of shoots in tomato plants grown in soil treated with the composition of the present invention at a concentration of 10 ml per liter soil.

Table 8 and FIG. 1B show that all treatments resulted in a decrease in the galling index in the roots, with a dose-related response for the composition of the present invention. The insecticide activity of the composition of the present invention at the highest dose was approximately equivalent to that of Nemacur EC400, with similar amounts of eggs found in treated plants (Table 9 and FIG. 1C).

### Example 4

### Effect on Common Red Mite in Greenhouse Cucumbers

The effect of the formulation of the present invention, Formulation 1, on common red mite (Tetranychus cinnabarinus) was tested on greenhouse cucumbers.

Brand IV 36 cucumbers were used, planted March 31, with planting gaps of 0.4 m in a row in flowerbeds of width 1.8 m, watered by dripping.

For each test, 5 treatments were used, with 4 replicates. The formulation was sprayed onto the plants using motorized bellows, with spray volume 45 liter/min, on June 18, 21 and 24.

Formulation 1 was sprayed onto plants at concentrations of 1%, 1.5%, 3%. A commercial product, Neemgard (Certis, USA), comprising 97% neem oil, was used as a standard.

For each replicate, 15 leaves of the same physiological age from height of 1.2 m above the ground were sampled from 10 different plants, from both sides of the flowerbed, and the number of adult mites on the lower surface of the leave was counted.

Statistical analysis was performed with OPEN-Start computer program with the Oneway Analysis of Variance procedure and Neuman-Keuls Test. Significance level of alpha selected=0.05.

A significant effect on reduction of mites by Formulation 1 was observed 5 days after the first spraying and second days after the second spraying. Formulation 1, at all concentrations tested, was found to be significantly more effective against red mite than the control or Neemgard.

Formulation 1, at concentrations of 1%, 2% and 3%, demonstrated reduction of red mites to levels of 1.67, 1.47 and 2.2 per leaf, respectively. The standard Neemguard treatment reduced levels to 7.47 mites per leaf. The control resulted in increased numbers of mites (11.27 per leaf).

Nine days after the third spraying, the results of red mite control by the test composition at 1%, 2% and 3% concentration was similar to that with Neemguard 1% (1.3, 1.1, 0.3 and 2.4 mites per leaf, respectively). The treatments resulted in a statistically significant decrease of mites per leaf as compared to control (18 mites per leaf).

Results are presented in Table 10, showing the average number of adult mites per cucumber leaf.

TABLE 10

| | | Inspection dates | | | |
|---|---|---|---|---|---|
| | | | June 23^{rd} | June 24^{th} | July 2^{nd} |
| | | June 18^{th} | +2 from | +3 from | +9 from |
| No. | Treatments | Zero count | 2^{nd} spray | 2^{nd} spray | 3^{rd} spray |
| 1 | Formulation 1 | 5.37 | 1.67 a | 1.33 a | 1.3 a |
| | 1% | | | | |
| 2 | Formulation 1 | 2.33 | 1.47 aR | 1.07 a | 1.1 a |
| | 2% | | | | |
| 3 | Formulation 1 | 10.8 | 2.20 a | 0.47 a | 0.3 a |
| | 3% | | | | |
| 4 | Neemguard 1% | 5.33 | 7.47 b | 2.40 a | 2.4 a |
| 5 | Control | 6.13 | 11.27 c | 18.27 b | 18.0 b |

Numbers followed by different letters on the same column are statistically different at the level of p = 0.05 according to Neuman-Keuls Test.

No phytotoxic damage to foliage, blossom, immature fruits, and ripe fruits of greenhouse cucumbers was observed after treatments with Formulation 1 at 2% or 3% concentrations, even after 3 successive sprayings.

It is concluded that Formulation 1 at concentrations of 1%, 2% and 3%, in spray volume of 45 liter/min exterminated the mite population on cucumbers significantly better than the control and to an extent comparable to Neemguard 1%.

### Example 5

### Effect on Red Spider Mite in Organically Grown Tomatoes

The effect of the formulation of the present invention on red spider mite (Tetranychus cinnabarinus) was tested on organically grown tomatoes.

Amielah 3060 brand tomatoes were used, planted May in a crossable tunnel with 50 mesh density. The tomatoes were planted in local soil, covered with gray-silver polyethylene, watered by drip irrigation. Flowerbed width was 1 m each.

The formulation was sprayed onto plants at concentrations of 1%, 1.5%, 3% on. August 27, and September 4, with a back motor bellow, liter/100 m² spray volume 5 with the addition of 0.5% ‘Egoz’ surface. Heliosulfur (1%) was used as a standard.

Each patch was 9 m long and 1 m wide. 4 replicates per test were used.

For each test, five leaves were collected from the upper third of the plant and the number of red spider mites counted.

Zero count was performed on August 25. Average infection level was 3.24 mites per leaf, with 62% of leaves infected. Counts were carried out on August 30, September 2, and September 7.

TABLE 11

| | | Average August 30^{th} | | Average September 2^{nd} | | Average September 7^{th} | |
|---|---|---|---|---|---|---|---|
| | Avg. on | Number | Level of | Number | Level of | Number | Level of |
| | Aug 25, | of mites | infected | of mites | infected | of mites | infected |
| | zero count | per leaf | leaves | per leaf | leaves | per leaf | leaves |
| Control | Mites | 3.65 a | 60% a | 3.85 a | 75% a | 11.4 a | 80% a |
| Formulation 1 | per leaf | 4.05 a | 55% a | 4.53 a | 70% a | 3.15 ab | 70% a |
| 3% | 3.24 | | | | | | |
| Formulation 1 | Infected | 3.53 a | 55% a | 3.30 a | 70% a | 1.66 b | 55% a |
| 1% | leaves | | | | | | |
| Formulation 1 | 62% | 2.35 a | 55% a | 3.15 a | 65% a | 1.40 b | 65% a |
| 0.5% | | | | | | | |
| Heliosulfur 1% | | 2.05 a | 65% a | 2.50 a | 60% a | 2.45 ab | 70% a |

* Different letters at the same column describe significant differences between treatments (p < 0.05), ANOVA according to Tukey-Kramer method.

No phytotoxicity was seen with any of the concentrations studied.

Formulation 1, at 1% and 0.5% were effective in controlling spider mites in trellising organic tomatoes, after two treatments. On the other hand, treatments with Heliosulfur and Formulation 1 3% did not yield results significantly different from the control after 2 treatments.

### Example 6

### Effect on Common Red Mite in Greenhouse Tomatoes

The effect of the formulation of the present invention on red spider mite (Tetranychus cinnabarinus) was tested on greenhouse tomatoes.

Ikram brand tomatoes were used, planted July 30, on sandy light soil, watered by drip irrigation. The greenhouse was made from a nylon cover 0.12 with net 50 mesh density. The area received with each watering 4 liter of Nugro fertilizer mixed with water on a 1:1 ratio. The greenhouse was sprayed every week with NeemGard mixed with Kusdaid (according to the recommended dosages).

For each test, 4 replicates were used. The formulation was sprayed onto the plants using motorized bellows, with spray volume 40 liter/min, on November 12 and 16.

The formulation was sprayed onto plants at concentrations of 1.5% and 3%, with Exhaust 0.5%. Neemgard (Certis, USA), 1%, comprising 97% neem oil, was used as a standard.

For each replicate, 25 leaves were collected from the upper third of the plant and the number of mites on the lower surface of the leaf was counted.

Results are shown in Table 12.

TABLE 12

| Treatment | Nov. 11, * | Nov. 16, ** | Nov. 19, |
|---|---|---|---|
| 1.5% Formulation 1 | 4.93 a | 3.00 a | 1.97 a |
| 3.0% Formulation 1 | 4.67 a | 2.35 a | 1.18 a |
| 1% NeemGuard | 5.30 a | 1.55 a | 2.05 a |
| Control | 4.97 a | 8.72 b | 6.62 b |

Identical letters mark insignificancy at the level of 99%.
* “Zero” estimation.
** Estimation prior to the second spraying.

The formulation of the present invention, at all concentrations tested, was found to be effective against red mite to a level comparable with that of Neemgard, and significantly more effective than control. No phytotoxicity was observed in any treatment.

### Example 7

### Effect on Melon Aphid in Greenhouse Cucumbers

The effect of the formulation of the present invention on melon aphid (Aphis gossypii) was tested on greenhouse cucumbers.

Cucumbers were planted on October 2, in local basalt soil, with planting gaps of 0.4 m in a row, flowerbed width 1.8 m, watered by dripping.

For each test, 4 treatments were used, with 4 replicates. The formulation was sprayed onto the plants using motorized bellows, with spray volume 50 liter/min, on March 23 and 26.

Formulation 1 was sprayed onto plants at concentrations of 1%, 1.5%, 3%. A commercial product, Timor C, comprising 0.75% matrine, 0.38% oxymatrine, 16.5% tea tree oil, 0.38% natural pyrethrin (Biomor, Israel) at 0.5% was used as standard.

For each replicate, 15 leaves of the same physiological age, at height 1-1.2 m above the ground, were collected from 10 different plants, on both sides of the central flowerbed. The number of aphids on the lower surfaces of each leaf was counted.

Oneway ANOVA was performed on the average number of melon aphids per cucumber leaf.

At the beginning of this study, the level of the aphid population was very high despite the release and spread of parasitoid wasps BioAphdius 500. This caused an immense damage to the cucumber plants. In addition to the aphids on the leaves, young fruits and stem, “clouds” of winged aphids were observed flying around the greenhouse, some of which settled on the nylon and on the mesh.

Formulation 1 according to at least some embodiments of the present invention, at all concentrations tested (1%, 1.5%, and 3%), was found to produce a significant decrease in the number of aphids within 3 days of treatment. A concentration of 3% was found to be more effective than 1% (1.8 aphids per leaf for 3%, 6.1 aphids per leaf for 1%). 4.8% aphids per leaf were counted for Formulation 1 1.5%. The formulations of the present invention were found to be more effective than the commercial product, Timor C.

Winged aphids returned to some of the leaves, and at concentration of 1% a few spawn were seen next to the adult aphids. At concentrations of 1.5% and 3% there were somewhat less winged aphids, and no spawn.

Due to the population load, it was decided to spray again after 3 days to try to obtain better pest control for a longer period of time. 3 days after the additional spraying action, a considerable rise in the number of aphids in the plants treated with control and 0.5% Timor C was observed. There were no significant differences among the Formulation 1 treatments. Once again, few spawns were observed in the Formulation 1 1% next to the winged aphids, while for the treatments of 1.5% and 3% no spawns were observed. At this time there were no significant difference between the Formulation 1 treatments.

One week from the second spray, on April 4, a considerable rise was observed in the aphid population in the control and 0.5% Timor C treatments (635.5 and 540 respectively). The population treated with Formulation 1 demonstrated a small rise only. The 1% Formulation 1 treatment resulted in 31.8 aphids per leaf including new spawns. The 1.5% Formulation 1 treatment showed only 10 aphids per leaf with only a small number of new spawn. The 3% Formulation 1 treatment demonstrated a reduction to 1.9 aphids per leaf without any new spawns (only winged adults). A significant difference was found between the 1% to the 3% dosages. The dosage 1.5% did not statistically differ from the 3% and the 1% dosages.

Results are shown in Table 13.

TABLE 13

| | | Inspections dates | | | |
|---|---|---|---|---|---|
| | | March 3 | March 26 | March 29 | April 4 |
| | | Zero | +3 of | +3 of | +7 of |
| No. | Treatments | count | 1^{st} spray | 2^{nd} spray | 2^{nd} spray |
| 1 | Formulation 1 | 127.5 | 6.1 c a* | 14.6 c | 31.8 b a* |
| | 1.0% | | | | |
| 2 | Formulation 1 | 137.2 | 4.8 c ab | 5.3 c | 10.0 b ab |
| | 1.5% | | | | |
| 3 | Formulation 1 | 123.3 | 1.8 c b | 2.4 c | 1.9 b b |
| | 3.0 % | | | | |
| 4 | Timor C 0.5% | 134.8 | 59.4 b — | 169.3 b | 540.0 a — |
| 5 | Control | 151.7 | 120.4 a — | 313.2 a | 635.5 a — |

Numbers followed by different letters are statistically different at the level of p = 0.05 according to Tukey-Kramer HSD
*After neutralizing the statistical effect of the control and the standard treatment (Timor C) there was a significant different among the Formulation 1 treatment.

Phytotoxicity tests showed that the control agent Formulation 1 is safe for use on greenhouse cucumbers up to a dosage of 3% and with a spray volume of 50 liter/min. No burn marks were observed on the leaves, flowers, young fruits or fruits.

It was concluded that all the Formulation 1 treatments (dosages 1%, 1.5% and 3%) at spray volume of 50 liter/min exterminate the melon aphid in cucumbers significantly better than the control and Timor C 0.5% treatments. Among the Formulation 1 treatments alone, the 3% Formulation 1 was significantly better than the 1% Formulation 1. 1.5% Formulation 1 did not significantly differ from the 3% Formulation 1 and the 1% Formulation 1.

In a further set of experiments, cucumbers planted on April 20, in marlstone loose soil were used. Planting gap was 0.4 m in a row, flowerbed width 1.8 m, fruit picking vegetation at 1.6 m, watered by dripping.

Four treatments were used, with 4 replicates. Replicate size was 54 m². (1.8 m×15 m)+partitioning row from each side. Spraying was done on June 15 and 16, with motor back bellows at spray volume: 35-40 liter/min.

1.5% and 3% Formulation 1 were sprayed, with water cannon with Neem oil plus Pyrethrum Natural made (Certis USA) and Knima, containing potassium salts (Certis USA) as standard.

Counting was performed on June 15, (zero count), June 16 and June 21,

For each replicate, 10 leaves of the same physiological age were sampled. The leaves were all sampled from height of 1.2 m above the ground, from 10 different plants, of both sides of the central flowerbed. On each leaf, all aphids were counted over the entire leaf surface.

Oneway ANOVA was performed on the average number of melon aphids per cucumber leaf.

Results are shown in Table 14.

TABLE 14

| | | Inspections' timings | | |
|---|---|---|---|---|
| | | June 15, | June 16, | June 21, |
| Number | Treatments | Zero count | +1 spray 1 | +5 spray 2 |
| 1 | Formulation 1 | 32.9 | 12.8 b | 3.4 c |
| | 1.5% | | | |
| 2 | Canon 0.4% + | 33.5 | 33.2 a | 28.4 b |
| | Knimat 1% | | | |
| 3 | Control | 31.3 | 36.6 a | 41.8 a |

Numbers following with different letters on the same column are statistically significant different (p = 0.05) according to Student's T test.

By the next day after the first treatment, a significant reduction in the number of melon aphids was noticed in the 1.5% Formulation 1 compared to the control and the canon+Knimat (12.8 aphids per leaf compared to 36.6 and 33.2 aphids per leaf respectively).

An excellent control result was achieved after an additional spray with Formulation 1 1.5%, a reduction to 3.4 aphids per leaf. Canon+Knimat achieved aphid reduction of 28.4. The control average results were 41.8 aphids per leaf. The control results achieved with Formulation 1 against melon aphids was statistically significantly different from both the control and the standard canon+Knimat treatments.

Leaf burning was observed only after spraying double concentration of Formulation 1, 3%. No burn marks were observed on flowers, immature and ripe fruits. Even after two consecutive spraying of Formulation 1 1.5%, at 24 hours interval, no burning whatsoever were observed.

It was concluded that treatment with 1.5% Formulation 1 (40 liter/min.) exterminated melon aphids on cucumbers significantly more efficiently than the control or the canon+Knimat treatments.

Treatment with Formulation 1 is safe to use on greenhouse cucumbers with 1.5% Formulation 1 (40 liter/min.) even with two consecutive spraying at 24 hours interval.

### Example 8

### Effect on Tobacco Whiteflies Larvae in Organically Grown Tomatoes

The effect of the formulation of the present invention on tobacco whiteflies larvae was tested on organically grown tomatoes.

Organic tomatoes, Amielah 3060 brand were used, planted in May, in crossable tunnels on local soil, covered with a silver-gray polyethylene, rocky soil, in 10 m×100 m structures with netting of 50 holes per inch², with drip irrigation.

Plants were treated regularly during the period prior to this study with Ganikan 1%, Totach 0.4% against tobacco whiteflies, with Heliosulfure 1% cc/min against the fungus oidium and spider mites. Commercial spraying was carried out by back bellows, with spray volume of 30 liter/min. The patch was chosen for this study after the pest started to develop and establish in the area.

Formulation 1 was sprayed onto plants at concentrations of 1%, 1.5%, 3%, using motor back bellows with spraying volume of 50 liter/minute on August 27. A commercial product, Ganikan, comprising Neem oil and vegetable oil was used as a standard. On September 4, the patch was sprayed again, with the addition of ‘Egzoz’ surface 0.5%.

For each test, 4 replicates were used.

Zero counting was performed on August 25, before the study began. Five leaves from the upper third of the plant were sampled when each leaf is structured from 3-5 leaflets. Sampling average was 9.46 larvae per leaf. 36% of the leaves were infested.

Later on, 5 leaves from the center of the plant were sampled. Leaves were picked from the upper third of the plant. These leave were tested for the presence of tobacco whiteflies larvae and pupae. These assessments were preformed on August 30, September 2 and 7.

TABLE 15

| | | Aug 30^{th} count | | Sep 2^{nd} count | | Sep 7^{th} count | |
|---|---|---|---|---|---|---|---|
| | | Avg. | Rate of | Avg. | Rate of | Avg. | Rate of |
| | Zero count | # pests | infested | # pests | infested | # pests | infested |
| Treatment | Aug 25 | per leaf | leaves | per leaf | leaves | per leaf | leaves |
| Control | Avg. # | 9.90 A | 60% A | 9.75 A | 85% A | 14.20 A | 95% A |
| Formulation 1 | pests | 0.45 A | 15% A | 5.00 A | 75% A | 3.13 B | 70% A |
| 3% | 9.46 | | | | | | |
| Formulation 1 | Infested | 0.80 A | 50% A | 5.46 A | 85% A | 3.60 B | 55% A |
| 1% | leaves | | | | | | |
| Formulation 1 | 36% | 1.00 A | 25% A | 4.20 A | 65% AB | 2.50 B | 55% A |
| 0.5% | | | | | | | |
| Ganiken 1% | | 2.20 A | 50% A | 0.35 A | 20% B | 1.10 B | 45% A |

Results are shown in Table 15.
* Different letters at the same column describe significant differences between treatments at the level of p < 0.05 according to ANOVA by Student**/Tukey -Kramer.

No phytotoxicity was found with any of the concentrations tested.

The formulation of the present invention, particularly at 1% and 1.5%, were found to be effective against tobacco aphid larvae to an extent comparable to that of the standard.

The study started with medium-high pest density, for adult activity and ovipositioning. The (pest) populations developed mainly in the control patches—in most counts the treatments resulted in significantly reduced pest numbers compared to control. Formulation 1 at 1%-0.5% appears to be efficient throughout the study, with results comparable to those for Ganikan.

During the count that was performed on September 2 it appeared that the activity of some of the agents has ceased, or that they were not acting on the young plants. The additional treatment on September 4 extremely improved the pesticide results, and succeeded in dramatically reducing the average number of pests per leave.

### Example 9

### Effect on Tobacco Whitefly Larvae in Organically Grown Eggplants

The effect of the formulation of the present invention on tobacco whitefly (Bemisia tabaci) larvae was tested on organically grown eggplants.

Eggplant strain margarita were used, planted on September 17, in crossable tunnels in local soil, using 10 m×100 m structures with polyethylene, watered by drip irrigation.

The formulation was sprayed onto plants at concentrations of 0.75%, 1.5% and 3%, with motor back spray, with spray volume 15 liter/minute, on November 12 and 16, with the addition of ‘Egoz’ surface 0.5%. A commercial product, Zohar LQ215, comprising 170 g fatty acid potassium salt per liter detergent (Zohar Dahlia Ltd, Israel), at 0.4% with Nimex 45 (azadirachtin) was used as a standard.

Zero counting was performed on November 10, before the study began. Five leaves from the center of the plant were sampled in each patch. Sampling average was 4.30 eggs per leaf and 3.15 larvae per leaf.

Later on, 5 leaves from the center of the plant were sampled in each patch. These leaves were tested for the presence of tobacco whiteflies adults, eggs, larvae and pupae. These assessments were preformed on November 15, 20 and 24.

Results are shown in Table 16.

TABLE 16

| | | Count on Nov 15^{th} | | | Count on Nov 20^{th} | | | Count on Nov 24^{th} | | |
|---|---|---|---|---|---|---|---|---|---|---|
| Treatment | Zzero | larvae | egg | adult | larvae | egg | adult | larvae | egg | adult |
| Control | Larvae | 6.9 | 4.5 AB | 0.1 | 16.0 | 3.95 | 0.15 | 18.6 | 2.50 | 0.15 |
| Formulation 1 | 3.15 | 7.6 | 4.5 AB | 0.0 | 9.95 | 2.40 | 0.10 | 16.8 | 2.60 | 0.00 |
| 3% | Egg | | | | | | | | | |
| Formulation 1 | 4.30 | 6.0 | 2.0 B | 0.0 | 11.4 | 1.90 | 0.10 | 13.8 | 1.85 | 0.05 |
| 1.5% | | | | | | | | | | |
| Formulation 1 | | 9.8 | 3.8 AB | 0.1 | 9.35 | 2.15 | 0.05 | 17.1 | 1.00 | 0.00 |
| 0.75% | | | | | | | | | | |
| Nimex 45 + | | 10.4 | 7.9 A | 0.0 | 11.8 | 3.70 | 0.00 | 16.7 | 2.30 | 0.00 |
| Zohar LQ215 | | | | | | | | | | |

* Different letters at the same column describe significant differences between treatments at the level of p < 0.05 according to ANOVA by Student.

No phytotoxicity was observed at any of the concentrations studied.

The study started with low-medium pest density, for adult activity, ovipositioning and beginning of establishment. Treatments with Formulation 1 appear to be efficient throughout the study. Furthermore, results after treatment with 1.5% concentration were significantly better than the commercial treatment for average egg measurement per leaf on the November 15 count, after one treatment.

Nimex, with the addition of LQ215, is efficient on adults and larvae, but is not very efficient against eggs on the contaminated hairy leaves of the eggplants in the sample.

During the November 15 count, it seems that some of the treatments, in some of the measurements achieved limited activity compared to the control. The additional treatment on November 16 improved considerably the treatment effect, and results with significant reduction in average number of individuals per leaf, in the eggs and adults measurements. A slightly lesser effect was noticed for the larvae.

It is very important to note that the hairy eggplant leaves and the weather conditions were limiting factors for this study. The hairs on the leaves negatively affect the control-agent penetration, and low temperatures disrupt and slow down the pest and the control agent activities.

In an addition set of tests, Santos brand eggplants were used, planted September 27, in heavy soil, with planting space 0.4 among seedling, 2 rows in a flowerbed, flowerbed width 1.8 m. Watering was by drip irrigation. The plants were at the stage of flowers and young fruit, with plants at 15 cm height.

Four treatments were used, with 4 replicates. Replicate size 36 m² (a row of 10 m+2 barrier rows of each side of the central row).

Spraying was performed on April 9 and 13, using motor back-spray with air pressure force and spray volume 50 liter/min. 1.5% and 3% Formulation 1 sprayed, with Zohar LQ215, containing 170 gr/liter detergent fatty acid potassium salt, (Zohar Dalia Ltd) as standard.

Counts were performed on April 9 (zero count), 13, 16 and 23. For each count, 15 leaves at the peak of their growth were randomly sampled. Leaves were all at the same height of 1 meter above the ground. The last leave count was performed one level above that. Mobile nymphs, immobile nymphs and pupae were counted.

One-way analysis of variance (ANOVA) was performed on the average numbers of whiteflies per eggplant leaf. Mobile nymphs, immobile nymphs and pupae were counted separately from the adults.

Results are shown in Tables 17 and 18.

TABLE 17

| | | Number of whiteflies immature in average | | | |
|---|---|---|---|---|---|
| | | per leaf during the different inspections | | | |
| | | (days from last implementation) | | | |
| | | April 9, | April 13, | April 16, | April 23, |
| No. | Treatments | Zero count | (+5) | (+3) | (+10) |
| 1 | Formulation 1 | 4.8 | 2.8 b | 1.7 b | 0.2 b b* |
| | 1.5% | | | | |
| 2 | Formulation 1 | 5.0 | 2.7 b | 1.1 b | 0.2 b b* |
| | 3% | | | | |
| 3 | LQ215 0.4% | 5.6 | 3.5 b | 3.3 b | 4.8 b c* |
| 4 | Control | 5.3 | 6.8 a | 8.4 a | 33.7 a — |

Numbers followed by different letters are statistically different at the level p = 0.05 according to Tukey-Kramer HSD.
*Significance test without the control.

TABLE 18

| | | Number of whiteflies adults in average | | | |
|---|---|---|---|---|---|
| | | per leaf during the different inspections | | | |
| | | (days from last implementation) | | | |
| | | April 9, | April 13, | April 16, | April 23, |
| No. | Treatments | Zero count | (+5) | (+3) | (+10) |
| 1 | Formulation 1 | 4.3 | 0.4 b | 0.7 c | 1.0 c |
| | 1.5% | | | | |
| 2 | Formulation 1 | 4.5 | 0.2 b | 0.5 c | 1.3 c |
| | 3% | | | | |
| 3 | LQ215 0.4% | 4.5 | 0.9 b | 1.9 b | 4.4 b |
| 4 | Control | 5.1 | 4.9 a | 6.7 a | 18.8 a |

Numbers followed by different letters are statistically different at the level p = 0.05 according to Tukey-Kramer HSD.

Treatments started when the whitefly population was at the level of 4.8 to 5.6 average mobile nymphs, immobile nymphs and pupae per leaf and 4.3 to 5.3 adults per leaf. Five days after the first spray, the Formulation 1 (1.5% and 3%) demonstrated complete mobile larvae extermination while the LQ215 0.4% treatment had few survivals. During the general count of mobile nymph, immobile nymph and pupae, a reduction to 2.8 and 2.7 immobile nymphs and pupae pear leaf, respectively, was found with Formulation 1. The LQ215 0.4% demonstrated reduction only to 3.5 nymphs and pupae per leaf. The control showed an increase to 6.8 mobile nymphs, immobile nymphs and pupae per leaf was noticed. At this time all treatments were significantly different from the control similar to each other. The number of adults after the two Formulation 1 treatments was reduced almost entirely to 0.4 and 0.2 adult per leaf, and to 0.9 adults per leaf in the LQ215 0.4%. The control had 4.9 adults per leaf and was significantly different than the other treatments.

Three days after the second spray, an additional reduction in immobile nymphs was noticed with Formulation 1 1.5% and 3%, to the levels of 1.7 and 1.1 per leaf, respectively. With LQ 215 0.4%, the average number of immobile nymphs remained 3.3 per leaf. The control demonstrated at this time an increase to 8.4 nymphs per leaf and was statistically significantly different from the other treatments. The different treatments did not significantly differ from each other.

The number of adults after the second spray remained low in the Formulation 1 1.5% and 3% groups, and was 0.7 and 0.5 adults per leaf on average and did not significantly differ from each other. The LQ215 0.4% group demonstrated a small increase in adults number, 1.9 per leaf. This treatment (LQ215 0.4%) was of inferior quality compared to Formulation 1, and was statistically different from both Formulation 1 treatment and from the control. The control had an increase to a level of 6.7 adult per leaf.

Ten days after the second spray (15 days from the first spray), the control group showed a dramatic increase in the number of immobile nymphs to 33.7 nymphs per leaf. The Formulation 1 1.5% and 3% groups had a reduction to 0.2 nymphs per leaf. The standard LQ215 0.4% demonstrated a small increase to the level of 4.8 nymphs per leaf. All the treatments (Formulation 1 1.5% and 3% and LQ215 0.4%) were statistically significantly different from the control. When the treatments were compared among themselves without the control, the Formulation 1 treatment were significantly more effective than the standard LQ215 0.4% treatment.

A dramatic increase in the number of adults was demonstrated in the control up to the average of 18.8 adults per leaf. In the Formulation 1 1.5% and 3% and LQ215 0.4% a small increase to the level of 1.0, 1.3 and 4.4 adults per leaf accordingly. The efficiency of all treatment was different from the untreated control and the Formulation 1 was more efficient compare to the standard.

In the Formulation 1 treatment with double concentration, 3%, no burn were noticed on flowers, young fruits, and fruits. This control agent is safe to be used on eggplants.

It is concluded that Formulation 1 at concentrations of 1.5% and 3% with spray volume of 50 liter/min is effective in exterminating mobile nymphs, immobile nymphs, pupae and adults of tobacco whitefly, to a level comparable to that of standard agent LQ215 0.4% and even better towards the end of this study.

### Example 10

### Effect on Western Flower Thrips in Eggplants

The effect of the formulation of the present invention on Western flower thrips (Frankliniella occidentalis) was tested on eggplants.

Margaria brand eggplants were used, planted on September 17, in local soil, in a crossable tunnel, with roof and walls covered with polyethylene. The plants were watered by drip irrigation. Seven flowerbeds, each 100 m long were used.

The formulation was sprayed onto plants at concentrations of 0.75%, 1.5%, 3%, with a commercial composition, Neemex 45 (azadurachtin fatty acid) plus Zohar LQ215 as standard.

Zero count was carried out on November 10.

The plants were sprayed with 0.75%, 1.5% and 3% Formulation 1, on November 12 and 16, with motorized bellows at spray volume 15 liter/minute. Nimex 45 (0.1%)+Zohar LQ215 was used as standard.

4 replicates were used. For each replicate, 5 eggplant leaves were collected from the center of the plant and the number of thrips per leaf counted.

Counting was performed on November 15, 20, and 24.

Results are shown in Table 19.

TABLE 19

| | Average number of thrips per leaf * | | | |
|---|---|---|---|---|
| | Zero count | | | |
| | Nov 10 | Nov 15^{th} | Nov 20^{th} | Nov 24^{th} |
| Control | 0.55 | 0.2 | 0.05 ab | 0.3 a |
| Formulation 1 | | 0.1 | 0.00 b | 0.0 b |
| 3% | | | | |
| Formulation 1 | | 0.0 | 0.00 b | 0.1 ab |
| 1.5% | | | | |
| Formulation 1 | | 0.1 | 0.1 ab | 0.05 ab |
| 0.75% | | | | |
| Nimex 45 + | | 0.0 | 0.2 a | 0.3 a |
| Zohar LQ215 | | | | |

* Different letters at the same column describe significant differences between treatments at the level of p < 0.05 according to ANOVA by Student.

The formulation of the present invention, at 3% was found to be effective against the thrips to an extent comparable to that of the standard. Concentrations of 1.5% and 0.75% were found to be slightly less effective than the commercial product, but the difference was not statistically significant.

No phytotoxicity was seen with any of the concentrations studied.

### Example 11

### Effect on European Mite in Apples

The effect of the formulation of the present invention on European mites (panonychus ulmi) was tested on apples.

Granny Smith brand apples were used, planted winter, 1991. Trees were at the height of 3.0 meters, at the end of the blossom stage, beginning of petals, with young fruits just starting to show. Trees were watered by dripping. Soil was Rocky Terra-Rosa.

For each test, 6 treatments were used, including 1 safety replicate, with 3 replicates. The formulation was sprayed onto the plants using a spray gun, with spray pressure of 25 atmospheres, on April 18 and 21.

Formulation 1 was sprayed onto trees at concentrations of 0.75%, 1%, 1.5%, and 3%, with an additional spraying after 3 days for 0.75% and 1.5% concentrations. A commercial composition, EOS oil comprising 99% mineral oil (822 g/l) (SK Corporation, Korea) as standard.

Inspection was carried out on April 12, (zero count); April 21, (+3 from first spray); April 26, (+8 from first spray and +5 from second spray); and April 30, (+12 from first spray and +9 from second spray).

For each replicate, 15 leaves were sampled from both sides of the central tree. The sampled leaf was the oldest of its leaf rosette. Adult mites were counted on both sides of the leaf.

One-way analysis of variance (ANOVA) was performed over the average number results of adult mite per leaf among the different treatments in each count.

Results are shown in Table 20.

Initially, infection by European Mites was between 4.8 to 7.2 mites per leaf. Three days after spraying, all treatments demonstrated extermination of adults mites (less than 1 mite per leaf) compared with the control that had 6.6 mites per leaf.

In order to compare a single Formulation 1 treatment to a double Formulation 1 treatment, an additional spray was performed 3 days after the first Formulation 1 treatment for concentrations of 1.5% and 0.75% Formulation 1.

Eight days after the first spray with Formulation 1 1.5% (a single treatment), a reduction in the quantity of mites per leaf towards 0.65 mites per leaf was noticed. Similar results were demonstrated with the double treatment of 0.75% Formulation 1. The double treatment with Formulation 1 1.5% demonstrated a strong reduction to 0.3 mites per leaf. At this time point, the differences between the different Formulation 1 treatments were not statistically significant, and even the result for standard EOS 1% with (0.2 mites per leaf) was not statistically significant different from that of any of the Formulation 1 treatments. All Formulation 1 treatments and the standard EOS treatment were statistically significant different from the control, in which an increase to 8.3 mites per leaf was demonstrated.

A significant increase of mites to 15.1 mites per leaf was demonstrated in the control 12 days after the first spray. Leaves treated with control changed color to bronze due to the effect of the mites. In the double spray of 0.75% Formulation 1 1.82 mites per leaf were counted, in the single and double spray of 1.5% Formulation 1 3.23 and 0.95 mites per leaf, respectively, were counted. All the spray treatments are significantly different from the control and are not different from the standard EOS 1%, which had 1.2 mites per leaf. The single replicate (for safety only) of 3% Formulation 1 had 0.8 mites per leaf. This double concentration did not demonstrate any phyto-toxic signs on the young fruits nor on the leaves.

TABLE 20

| | | Number of Adult mites per leaf | | | |
|---|---|---|---|---|---|
| | | | | | April 30^{th} - |
| | | April 18^{th} | April 21^{st} | April 26^{th} | 05-12 +12 or |
| | | Zero | +3 from | +8 or +5 | +9 from |
| No. | Treatments | count | spray | from spray | spray |
| 1 | Formulation 1 | 5.4 | 0.93 b | 0.65 b | 1.82 b |
| | 0.75% × 2 | | | | |
| 2 | Formulation 1 | 5.0 | 0.78 b | 0.65 b | 3.23 b |
| | 1.5% | | | | |
| 3 | Formulation 1 | 4.8 | 0.82 b | 0.3 b | 0.95 b |
| | 1.5% × 2 | | | | |
| 4 | EOS 1% | 7.2 | 0.30 b | 0.2 b | 1.20 b |
| 5 | Control | 5.6 | 6.60 a | 8.3 a | 15.1 a |
| 6 | Formulation 1 | 5.7 | 0.4 | 0.1 | 0.8 |
| | 3% (Single | | | | |
| | replicate) | | | | |

Numbers following with different letters are statistically significant different at the level of p = 0.05 according tp Tukey-Kramer HSD

Formulation 1 3% did not damage the leaves, blossom or young fruits.

It is concluded that treatment with Formulation 1 at a concentration of 0.75% with double spraying is effective in controlling European mite in apples and achieved similar results to the standard EOS 1% treatment. Treatments with Formulation 1 at a concentration of 1.5%, single and double spraying, are effective in controlling European mite in apples and achieved similar results to the standard EOS 1% treatment.

In a second set of experiments, the effect of Formulation 1 was studied on Star-King brand apples, from trees 2.5 m tall, planted winter 1999, in top and clay soil. The trees were still growing, bearing fruit of 35 mm diameter, watered by drip irrigation.

3 treatments were used, with 3 replicates. Formulation 1 at concentrations 1.5% and 3% was sprayed using motorized back bellows, with spray volume by seepage (about 180 liter/min), on July 10 and 14.

In each replicate 20 leaves were examined from both sides of the central tree. The counted leaves were fully grown on the lower part of the annual twig (the oldest leaves). All green apple aphids were counted.

Initially, numerous mites per twig were counted. Two days after spraying a considerable reduction was observed in the Formulation 1 treatments. On July 14, a second treatment was performed on 2 replicates of 1.5% Formulation 1 only. On July 17, a small increase in the number of adult mites was observed in all treatments. Young mites were also observed in the 3% Formulation 1 treatment and in the control.

Results are shown in Table 21

TABLE 21

| | | Number of adult mites per leaf | | | |
|---|---|---|---|---|---|
| | | July 10, | July 12, | July 14, | July 17, |
| No. | Treatments | Zero count | +2 | +4 | +7 |
| 1 | Formulation 1 | 37.5 | 5.5 | 5.5 | 7.5 |
| | 1.5% | | | | |
| 2 | Formulation 1 | 37.5 | 7.5 | 9 | 9.5 |
| | 3% | | | | |
| 3 | Control | 37.5 | 35 | 35 | 40 |

In all replicates with all dosages no leaf nor fruit burns were observed, even after spraying with double concentration of 3%.

It is concluded that treatment with the control agent Formulation 1 at concentrations of 1.5% and 3% is effective in controlling panonychus ulmi in apples.

### Example 12

### Effect on Green Apple Aphid

The effect of the formulation of the present invention on green apple aphid (aphis pomi) was tested.

Pink Lady brand apples were used, planted 2007. Trees were at the height of 2.5 meters, still growing, bearing fruits of 60 mm diameter. Soil was Basaltic ventilated.

For each test, 3 treatments were used, with 4 replicates. The formulation was sprayed onto the plants using motorized bellows, with spray volume by seepage (about 180 liter/min) on August 21 and 28.

The formulation was sprayed onto plants at concentrations 1.5% and 3%.

For each replicate, 20 leaves were examined from both sides of the central tree. All green apple aphids were counted.

One way analysis of variance (ANOVA) was performed on the average number of green apple aphids.

Initially, numerous green apple aphids were counted per twig. Four days after spraying, a considerable reduction was observed in the Formulation 1 treatments. On August 28, an additional spray was carried out.

Following the second spraying, a considerable pest reduction with both Formulation 1 (1.5% and 3%) treatments occurred. The average number of green apple aphid per leaf was 4.0 (1.5%) and 3.0 (3%), while the control demonstrated a small reduction only to 22.0 aphids per leaf.

Results are shown in Table 22.

TABLE 22

| | | Number of adult green apple aphid per leaf | | | |
|---|---|---|---|---|---|
| | | Aug. 21, | Aug. 25, | Aug. 28, | Sep. 1, |
| Number | Treatments | Zero count | +4 | +7 | +4 spray 2 |
| 1 | Formulation 1 | 35.0 | 18.0 b | 17.0 b | 4.0 b |
| | 1.5% | | | | |
| 2 | Formulation 1 | 35.0 | 16.0 b | 14.0 b | 3.0 b |
| | 3% | | | | |
| 3 | Control | 35.0 | 33.0 a | 30.0 a | 22.0 a |

Numbers following by different letters at the same column are statistically different at the level of p = 0.05 according to Tukey-Kramer HSD.

At all replicates with all dosages no leaf nor fruit burns were observed.

It was concluded that treatment with Formulation 1 at concentrations of 1.5% and 3% is effective in controlling the green apple aphid.

In a second set of tests, Golden brand apples were used, planted 1997, in Rocky Rendzina soil, with planting space of 3×4.5 m. Trees were at a height of about 2.5 m, still growing, with young fruits of diameter 8-12 mm. Watering was by drip irrigation.

Four treatments were used, with 4 replicates. Spraying was done by spray gun, with spray volume of about 180 liter/min, on April 23, 27, 30 and May 3. Formulation 1 at concentrations 0.75%, 1.5%, 3% were used. (3% with one replicate, only for safety purposes). Calypso 0.2% was used as standard.

For each replicate, 20 leaves were sampled from both sides of the central tree. All mites on the bottom side of the leaf were counted. All aphids on the bottom side of the leaf were counted.

One-way analysis of variance (ANOVA) was performed over the average number of aphids per leaf.

Results are shown in Table 23.

TABLE 23

| | | Number of adult green apple aphid per leaf | | | | |
|---|---|---|---|---|---|---|
| | | April 23^{rd} | April 27^{th} | April 30^{th} | May 3^{rd} | May 7^{th} |
| No. | Treatments | Zero count | +4 | +7 | +10 | +14 |
| 1 | Formulation 1 | 17.6 | 0.0 b | 0.0 b | 0.73 b a* | 0.65 b a* |
| | 0.75% | | | | | |
| 2 | Formulation 1 | 15.4 | 0.0 b | 0.0 b | 0.0 b b | 0.15 b b |
| | 1.5% | | | | | |
| 3 | Calypso 0.02% | 16.5 | 0.0 b | 0.0 b | 0.0 b b | 0.0 b b |
| 4 | Control | 20.6 | 29.7 a | 43.7 a | 37.3 a — | 49.3 a — |

Numbers followed by different letters are statistically different at the level p = 0.05 according to Tukey-Kramer HSD.
*Significance test without the control.

At the beginning of this study there were numerous green apple aphids, (between 15.4 to 20.6 aphids per leaf). Four days after spraying, a significant reduction in aphid number was demonstrated in the Formulation 1 (0.75% and 1.5%) treatments to zero aphids per leaf similar to the standard Calypso 0.02%. In the control the average numbers of aphids increased to 29.7 aphids per leaf.

Furthermore, one week after spraying, no new aphids were to be found on the leaves in the different Formulation 1 treatments as well as the standard Calypso. The numbers in the control increased again to 43.7 aphids per leaf.

Ten days after the spraying an establishment of un-winged aphid population could be found in the Formulation 1 0.75% treatment (nearly 1 aphid per leaf). The Formulation 1 1.5% and Calypso 0.02% treatments were clear of aphids, while the control had 37.3 aphids per leaf. Formulation 1 0.75% was significantly less effective when compared with the Formulation 1 1.5% and Calypso 0.02% treatments.

No effect was noticed on predatory mites in the Formulation 1 treatments. No leaf or fruit burns were noticed with Formulation 1 3%.

It was concluded that Formulation 1 at concentrations of 0.75% and 1.5% is efficient in exterminating green apple aphid, comparable to the standard Calypso 0.02%. 2.

### Example 13

### Effect on Wooly Apple Aphid

The effect of the formulation of the present invention on wooly apple aphid (Eriosoma lanigerum) was tested.

Starking brand apples were used, planted 1995, in an organic orchard, with drip irrigation. Trees were medium size, near harvest. Planting space was 2.5×4.5 m. Soil was toph and clay.

3 treatments were used, with 4 replicates per test.

The formulation was sprayed onto plants at concentrations of 1.5% and 3%, using motor back bellows, with seepage (spray volume about 180 liter/min), on August 21 and 28.

All the woolly apple aphid colonies were counted on 10 branches on the central tree in all replicates on the peripheral branches only.

One way analysis of variance (ANOVA) was performed on the average number of colonies of woolly apple aphids on the central tree in each replicate.

Results are shown in Table 24.

TABLE 24

| | | Number of colonies per 10 branches | | | |
|---|---|---|---|---|---|
| | | Aug. 21^{st}, | | | |
| | | Zero | Aug. 25^{th}, | Aug. 28^{th}, | Sep. 1^{st}, 2^{nd} |
| No. | Treatment | Count | +4 | +7 | spray +4 |
| 1 | Formulation 1 | 19 | 10 b | 12 b | 2 b |
| | 1.5% | | | | |
| 2 | Formulation 1 | 22 | 9 b | 9 b | 1 b |
| | 3% | | | | |
| 3 | Control | 23 | 22 a | 24 a | 25 a |

Numbers followed by different letter at the same column are statistically different at the level of p = 0.05 according to Tukey-Kramer HSD

At the beginning of the study (zero count), 19 to 23 woolly apple aphid colonies per 10 branches were observed. Four days after spraying, a 50% reduction of the number of living colonies was observed. About one week from application with only inconsiderable change, a second spray was performed on August 28. Four days later, a significant reduction in the number of living colonies to 1 or 2 woolly apple aphid colonies per 10 branches was found, compared with 25 colonies in the control. In each and every counting date the extermination efficiency was significantly better than the control with no significant difference between the Formulation 1 treatments.

No phytotoxic effect was observed.

The formulation of the present invention at concentrations of 1.5% and 3%, was found to be effective against wooly aphid in apples.

### Example 14

### Other Insect Pests

Formulation 1 was found to be effective against a number of insect pests, including Lissorhoptrus oryzophilus (rice water weevil), Tribolium castaneum (flour beetle), and Sitophilus oryzae (rice weevil). Formulation 1 was administered at a concentration of 1% (A), 2% (B) and 3% (C) stock solution in water, in the amount of 1 ml per kilogram of soil, in which each of the above insects was present (20 specimens were examined in each case). The results are given below, clearly indicating that Formulation 1 effectively killed these insects, in contrast to the control.

| | Treatment A | Control A | Treatment B | Control B | Treatment C | Control C |
|---|---|---|---|---|---|---|
| insect | (alive/dead) | (alive/dead) | (alive/dead) | (alive/dead) | (alive/dead) | (alive/dead) |
| oryzophilus | 0/20 | 13/7 | 0/20 | 18/2 | 0/20 | 14/6 |
| Tribolium | 0/20 | 20/0 | 0/20 | 17/3 | 0/20 | 18/2 |
| Sitophilus | 0/20 | 17/3 | 1/19 | 15/5 | 0/20 | 20/0 |

### Example 15

### Other Tests

Formulation 1 was found to be effective against a wide variety of insect species and other pests and diseases as summarized below.

### List of Insects and Mosquitoes Effectively Targeted by Formulation 1:

| | Insect | Type of test | Comment |
|---|---|---|---|
| | Harmful to people & public health | | |
| 1 | Scientific name: Culex pipiens | Field and | Exterminated larva and |
| | English name: House mosquito | laboratory experiments | pupa; repelled egg laying |
| 2 | Scientific name: Culex univittatus | Field and | Exterminated larva and |
| | English name: none | laboratory experiments | pupa; repelled egg laying |
| 3 | Scientific name: Aedes mariae | Observation | Exterminated larva and |
| | English name: none | | pupa |
| 4 | Scientific name: Cimex lectularius | Observation | |
| | English name: Bed bug | | |
| | Agricultural pests | | |
| 5 | Scientific name: Panonychus ulmi | Field experiment | Apple trees |
| | English name: European Red Mite | | |
| 6 | Scientific name: Tetranychus cinnabarinus | Field experiment | Hothouse cucumbers |
| | English name: Common red mite | | Hothouse tomatoes |
| 7 | Scientific name: Eutetranychus orientalis | Observation | |
| | English name: The oriental red mite | | |
| 8 | Scientific name: Polyphagotarsonemus latus | Observation | |
| | English name: Broad mite | | |
| 9 | Scientific name: Aphis pomi | Field experiment | Apple trees |
| | English name: Green apple aphid | | |
| 10 | Scientific name: Bemisia tabaci Gennadius | Field experiment | Eggplants, tomatoes |
| | English name: Tobacco whitefly | | |
| 11 | Scientific name: Eriosoma lanigerum | Field experiment | Apple trees |
| | English name: Woolly apple aphid | | |
| 12 | Scientific name: Aphis gossypii | Field experiment | Hothouse cucumbers |
| | English name: Melon Aphids | | |
| 13 | Scientific name: Liriomyza trifolii Burgess | Observation | |
| | English name: Serpentine leafminer | | |
| 14 | Scientific name: Tuta absoluta | Observation | |
| | English name: South American tomato leafminer | | |
| 15 | Scientific name: Rhynchophorus ferrugineus | Observation | Dates |
| | English name: Red Palm Weevil | | |
| 16 | Scientific name: Stephanitis pyri | Observation | |
| | English name: Pear Lace Bug | | |
| | Soil diseases | | |
| 17 | Nematode | Observation | |
| 18 | Rhizoctonia sp. fungi | Observation | |
| 19 | Verticillium sp. fungi | Observation | |
| 20 | Pythium sp. Fungi | Observation | |

Formulation 1 has proven its ability to exterminate a wide variety of pests, both insects and mites. Although a small number of species are not harmed by Formulation 1 (such as the predatory mite Amblyseius swirskii, and Phytoseiulus persimilis), most of the species harmed are small arthropods (including insects, and mites)

Therefore, there is a reasonable chance that Formulation 1 is harmful to additional species of small arthropods, both exterminating and repelling them. There are many pests for which extermination experiments are not sufficiently effective, for example, many species of fire ant including the small fire ant (also known as the electric ant) Wasmannia auropunctata that has invaded Israel recently. Two other fire ants (Solenopsis invicta and Solenopsis richter) have invaded the United States and are spreading there, in addition to the local species that have long been known there (such as Solenopsis xyloni and Solenopsis geminata). It seems that Formulation 1 will be effective against these species.

The mites that Formulation 1 was able to combat successfully are taxonomically close to ticks. There is a reasonable chance that Formulation 1 will be effective against ticks (perhaps requiring a slightly higher concentration because ticks are larger than mites).

Many human parasites, have parasitic traits similar to those of mosquitoes. Since Formulation 1 has a repellent effect, in addition to exterminating, it would be worthwhile to conduct in-depth testing of its effect on sand flies (Phlebotomus sp. and Sergentomyia sp.), black flies, (Simuliidae), midges (Ceratopogonidae) and other similar creatures.

Pine processionary moth larva (Thaumetopoea wilkinsoni) move exposed on pine branches and it is likely that they will be sensitive to spraying with Formulation 1.

In addition to these pests that are harmful to the public health, there are many agricultural pests that Formulation 1 might combat effectively, beyond those that have already been tested.

The efficacy of Formulation 1 has proven against many species of Acarida (such as: the European red mite [Panonychus ulmi], the oriental red mite [Eutetranychus orientalis], the common red mite [Tetranychus urticae] and the broad mite [Polyphagotarsonemus latus]. Therefore, it is reasonable that other vegetarian mites will also be harmed, such as flat mites, avocado or persea mites [Oligonychus perseae], rust mites, [Aceria anthocoptes], Tenuipalpus granati, and the two-spotted spider mite [Tetranychus uricae].

Similarly, it can be expected that Formulation 1, which has been proven effective in eradicating various aphids (such as the green apple aphid and the melon aphid [Aphis gossypii]), and whiteflies (Aleyrodoidea) (such as sweet potato whitefly [Bemisia tabaci]), will also be effective against other pests from these groups (for example, scale insects [Coccoidea]). Formulation 1 is likely to combat a range of aphids (Aphidoidae), whiteflies (Aleyrodoidea) and scale insects (Coccoidea) including red scale (Aonidiella aurantii), brown soft scale (Coccus hesperidium), peach trunk aphid (Pterochloroides persicae), Florida wax scale (Ceroplastes floridensis), mealy bugs (Planococcus), Aphis craccivora, wooly apple aphid (Eriosoma lanigerum) red date scale (Phoenicococcus marlatti) black olive scale (Saissetia oleae), bayberry whitefly (Parabemisia myricae), purple scale (Lepidosaphes beckii), pit scales (Asterolecamidae), parlatoria scale (Parlatoria), black parlatoria scale (Parlatoria pergandii.

Many varieties of moths and butterflies feed on leaves, flowers and other parts of the plants (for example: bordered straw moths [Heliothis peltigera], pink bollworm [Pectinophora gossypiella], Egyptian stemborer or Egyptian bollworm [Earias insulana], beet armyworm [Spodoptera exigua], African or Egyptian cotton leaf worm [Spodoptera littoralis], giant looper [Ascotis selenaria], Nychiodes palaestinensis, double-striped pug [Gymnoscelis rufifasciata], large white or cabbage butterfly [Pieris brassicae], small white [Artogeia rapae], pea blue or long-tailed blue [Lampides boeticus]). Similarly, many beetle types also feed on leaves and other plant parts (for example: lined click beetle or wireworm [Agriotes lineatus], various species of Alticinae and more); Formulation 1 can combat all of these pests.

Larvae of other beetle varieties are not commonly found on the surface of plants but rather harm plants by boring holes in their stems or branches (for example: Apate monachus, mango stem borer [Batocera rufimaculata] and Cerambyx dux, etc.). The larvae (grubs) of other varieties hide in the plants' roots and do their damage there (for example: the root borer [Oryctes agamemmon], Lixus anguinus, and the sugar beet or striped weevil [Lixus junci] and others.) Similarly concealed are the larvae of moths that feed on fruit and internal parts of the plant (for example: Deudorix livia, the European corn borer moth [Pyrausta nubilais], Etiella zinckenella, Arenipses sabella and the leopard moth [Zeuzera pyrina]). Many pesticides cannot reach pests in these hidden places. The repellent effect of Formulation 1 can be used against these pests even before the eggs are laid on the plants, which effectively prevent boring.

Several varieties of flies harm plants in ways similar to the moths and beetles listed above (for example: the onion maggot [Hylemyia antique], the olive fruit fly [Dacus oleae], and other fruit flies [Tephritidae], various types of Agromyzidae, Merodon geniculatus, and species of Drosophilidae). It is reasonable to assume that Formulation 1 will exterminate and/or repel them in a similar manner.

Furthermore formulation 5 was tested as for all of the above examples with formulation 1 and the same or similar results were obtained.

Hence all of the formulations according to various embodiments of the present invention are asserted to be useful against the above described pests, insects and diseases.

### Example 16

### Analysis of Essential Oils

Essential oils are extracts of plant materials and hence contain many important factors and ingredients, which relate to their efficacy in the described formulations. Without wishing to be limited in any way, several exemplary analyses of such essential oils are provided below; each of the components listed below, alone or in combination, also optionally form part of the formulations as described herein.

Lemongrass oil:

### GC Analysis (%):

### Geranial 38.84

### Neral 28.99

### Geraniol 6.58

### Geranyl Acetate 3.35

beta-Caryophyllene 2.08

### Methyl Heptenone 1.89

Terpinene-1-ol-4 1.83

### Linalool 1.27

### Camphene 1.10

### 4-Nonanone 0.92

### Limonene 0.61

### Caryophyllene Oxide 0.61

### Citronellal 0.57

cis-beta-Ocimene 0.52
trans-beta-Ocimene 0.31

### Borneol 0.30

### Citronellyl Acetate 0.23

beta-Pinene 0.21

### Myrcene 0.21

### Citronellol 0.20

alpha-Terpineol 0.19

### Terpinolene 0.07

para-Cymene 0.06

### Eugenol 0.04

Citronella Essential Oil

### Citronellal 32.15%

### Geraniol 21.07%

### Citronellol 12.69%

### Limonene 4.00%

Geranyl acetate 3.20%
Citronellyl acetate 2.83%

### Elemol 2.60%

### Germacrene D 2.25%

Delta cadinene 2.22%
Beta elemene 1.99%
Linalyl acetate 1.38%

### Eugenol 1.08%

### Linalool 0.97%

Neryl acetate 0.56%

### Geranial 0.48%

### Isopulegol 0.44%

### Neral 0.27%

### Nerol 0.22%

Beta phellandrene 0.11%

### Myrcene 0.10%

### Trans-trans-farnesol 0.08%

### Terpinolene 0.07%

### Alpha-pinene 0.03%

Tea Tree Oil

### Alpha-pinene 2.4%

### Sabinene 0.1%

### Alpha-terpinene 9.0%

### Limonene 0.9%

p-cymene 2.7%
1,8-cineole 2.2%
Gamma terpinene 20.0%

### Terpinolene 3.3%

### Terpinen-4-ol 40.8%

### Alpha-terpineol 2.7%

### Aromadendrene 1.6%

### Ledene 1.1%

### Delta-cadinene 1.3%

### Globulol 0.3%

### Viridiflorol 0.2%

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A natural pesticide formulation comprising tea tree oil in an amount of from about 5% to about 35% volume per volume over the volume of the formulation; an emulsifier in an amount of up to about 10% volume per volume; a vinegar in an amount of up to about 40% volume per volume and a further essential oil selected from the group consisting of citronella, rosemary oil, lemon grass oil and neem oil, each present in an amount of from about 5% to about 35% volume per volume, or a combination of two or more of said further essential oils; wherein a solvent carrier comprises the balance of the formulation.

2. The pesticide of claim 1, wherein said solvent carrier comprises water, and wherein said water is present in an amount of up to 70% volume per volume over the formulation.

3. The pesticide of claim 2, wherein said tea tree oil is present in an amount of from about 10% to about 22%, volume per volume.

4. The pesticide of claim 3, wherein said emulsifier is present in an amount up to about 3%, said vinegar is present in amount of up to about 10% and said further essential oil comprises citronella present in an amount of from about 10% to about 22%, volume per volume.

5. The pesticide of claim 3, wherein said further essential oil comprises lemon grass oil and rosemary oil.

6. The pesticide of claim 5, wherein said lemon grass oil is present in an amount up to about 10% volume per volume, said emulsifier is present in an amount up to about 3%, said vinegar is present in amount of up to about 5% and said rosemary oil is present in an amount of from about 10% to about 22%, volume per volume.

7. The pesticide of claim 3, wherein said further essential oil comprises citronella and neem oil.

8. The pesticide of claim 7, wherein said citronella is present in an amount of from about 10% to about 22%, volume per volume, said emulsifier is present in an amount up to about 3%, said vinegar is present in amount of up to about 10% and said neem oil is present in an amount up to about 5% volume per volume.

9. The pesticide of claim 3, wherein said further essential oil comprises rosemary oil, lemongrass oil and neem oil.

10. The pesticide of claim 9, wherein said rosemary oil is present in an amount of from about 10% to about 22%, volume per volume, said lemon grass oil is present in an amount of up to about 10%, said emulsifier is present in an amount up to about 3%, said vinegar is present in amount of up to about 5% and said neem oil is present in an amount up to about 5% volume per volume.

11. The pesticide of claim 1, wherein said further essential oil comprises citronella or rosemary oil, and said further essential oil is present in an amount equal to said tea tree oil.

12. A natural pesticide formulation, comprising Tea tree oil in an amount of from about 10% to about 22%, volume per volume over the volume of the formulation; Citronella in an amount of from about 10% to about 22%, volume per volume; Vinegar in an amount of up to 10% volume per volume; and an emulsifier in an amount of up to 3% volume per volume, with the remainder of the formulation comprising water.

13. A natural pesticide formulation, comprising Tea tree oil in an amount of from about 10% to about 22%, volume per volume over the volume of the formulation; Lemon grass oil in an amount up to 10%; Vinegar in an amount of up to 5% volume per volume; Rosemary oil in an amount of from about 10% to about 22%, volume per volume; and an emulsifier in an amount of up to 3% volume per volume, with the remainder of the formulation comprising water.

14. A natural pesticide formulation, comprising Tea tree oil in an amount of from about 10% to about 22%, volume per volume over the volume of the formulation; Citronella in an amount of from about 10% to about 22%, volume per volume; Vinegar in an amount of up to 10% volume per volume; neem oil in an amount of up to 5% volume per volume; and an emulsifier in an amount of up to 3% volume per volume, with the remainder of the formulation comprising water.

15. A natural pesticide formulation, comprising Tea tree oil in an amount of from about 10% to about 22%, volume per volume over the volume of the formulation; Lemon grass oil in an amount up to 10% volume per volume; Rosemary oil in an amount of from about 10% to about 22%, volume per volume; neem oil in an amount of up to 5% volume per volume; Vinegar in an amount of up to 5% volume per volume; and an emulsifier in an amount of up to 3% volume per volume, with the remainder of the formulation comprising water.

16. A natural pesticide formulation, comprising citronella in an amount of from about 10% to about 22%, volume per volume over the volume of the formulation; Lemon grass oil in an amount up to 10%; Vinegar in an amount of up to 5% volume per volume; Rosemary oil in an amount of from about 10% to about 22%, volume per volume; and an emulsifier in an amount of up to 3% volume per volume, with the remainder of the formulation comprising water.

17. The formulation of claim 1, wherein said emulsifier is selected from the group consisting of paraffin and polysorbate 20.

18. The formulation of claim 1, wherein said vinegar comprises apple vinegar.

19. The pesticide of claim 18, prepared as a dry formulation by adsorbing the formulation onto a solid carrier.

20. The pesticide of claim 19, wherein said solid carrier comprises an inert material and is selected from the group consisting of raw material, pellets, particles, microparticles and nano-particles.

21. The pesticide of claim 20, wherein said dry formulation is in a form selected from the group consisting of pellets, particles, microparticles and nano-particles.

22. The pesticide of claim 21, further comprising a dry diluent.

23. The pesticide of claim 1, further comprising a liquid diluent.

24. The pesticide of claim 23, wherein said liquid diluent dilutes the active formulation to form a total formulation such that the active formulation comprises from 0.2% to 6% of the total formulation.

25. The pesticide of claim 24, wherein said liquid diluent dilutes the active formulation to form a total formulation such that the active formulation comprises from 0.5% to 4% of the total formulation.

26. Use of the formulation of claim 1 for control of an insect infestation or agriculturally important diseases caused by at least one of a fungus, an oomycete, a bacterium, or a nematode, or a combination thereof.

27. Use of the formulation of claim 26, wherein said fungus is selected from the group consisting of Rhizoctonia, Pythium, Verticillium dahlia, and fusarium oxysporum.

28. Use of the formulation of claim 26, wherein said bacterium comprises streptomyces.

29. Use of the formulation of claim 26, wherein said insect infestation is selected from the group consisting of tetranychidae, aphididae, eriosomatidae, aleyrodidae, diaspididae, thysanoptera; pterygota; grain and seed crop pests, and agromyzidae.

30. Use of claim 29, wherein said tetranychidae are selected from the group consisting of spider mite, red spider mite, oriental red mite, fruit tree red spider mite, broad mite, and European red spider mite.

31. Use of claim 29, wherein said aphididae are selected from the group consisting of melon aphids, green apple aphids, and cotton aphids.

32. Use of claim 29, wherein said eriosomatidae comprise wooly aphids.

33. Use of claim 29, wherein said aleyrodidae comprise whitefly.

34. Use of claim 29 wherein said diaspididae comprise California red scale.

35. Use of claim 29, wherein said agromyzidae comprise serpentine leafminer or South American tomato leafminer.

36. Use of claim 29, wherein said pterygota comprise thrips.

37. Use of claim 29, wherein said grain and seed crop pests comprise one or more of members of the lissorphotrus family, members of the tribolium family, and members of the sitophilus family.

38. Use of claim 26, wherein the formulation is applied post-harvest to harvested plant materials.

39. Use of claim 26, comprising use as an insect repellent against insects harmful to public health or which are annoying to humans and/or animals.

40. Use of claim 39, wherein said insects comprise one or more of Culex pipiens, Culex univittatus, Aedes mariae, anopheles, blackfly, tsetse fly, Phlebotomus or Cimex lectularius.

41. Use of a natural insect control formulation for control of agriculturally important diseases, or as an insect repellent, wherein said formulation comprises vinegar, an emulsifier and an essential oil selected from the group consisting of citronella, rosemary oil, tea tree oil and lemon grass oil, in an aqueous carrier.

42. Use of claim 41, wherein said essential oils are present in a total amount of from 10% to 30% volume per volume over the volume of the formulation.

43. Use of claim 42, wherein said essential oils are diluted to a concentration of 0.001% to 3% before application.

44. Use of claim 43, comprising use as an insect repellent or insecticide against insects harmful to public health or which are annoying to humans and/or animals.

45. Use of claim 44, wherein said insects comprise one or more of Culex pipiens, Culex univittatus, Aedes mariae, anopheles, blackfly, tsetse fly, Phlebotomus or Cimex lectularius.

46. Use of claim 45, for application to a location selected from the group consisting of any type of outdoor location and any type of indoor location.

47. Use of claim 46, wherein said outdoor location includes one or more of agricultural fields, landscaped areas, other types of fields and grounds, ponds, lakes, rivers and other bodies of water, outdoor holding pens or coops for livestock, poultry.

48. Use of claim 46, wherein said indoor location includes one or more of hothouses, greenhouses, barns, chicken coops or barns, livestock pens.
